(19) United States
(12) Patent Application Publication
(10) Pub. No.: US 2010/0179565 A1
(43) Pub. Date: Jul. 15, 2010

(54) Rail for Delivering an Endovascular Stapler

(75) Inventors: Anne Brody Rubin, San Francisco, CA (US); Walter Bruszewski, Guerneville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/352,719

(22) Filed: Jan. 13, 2009

(51) Int. Cl. A61B 17/10

(52) U.S. Cl. 606/139; 227/175.1; 227/179.1; 227/181.1

(57) ABSTRACT

An endovascular stapler delivery apparatus having a continuous geometrically non-symmetrical delivery rail operable to guide a stapling device to one or more stapling locations within a body vessel is disclosed. The delivery rail includes a first elongated leg, a second elongated leg in parallel with the first leg, and a self-expanding distal loop extending between a distal end of the first leg and a distal end of the second leg. The distal loop self-expands at a delivery site within the vessel at an angle with a longitudinal axis of the vessel and includes at least first and second portions that abut the vessel at opposing and longitudinally offset locations of a wall of the vessel. The self-expanding distal loop operates to press and align the stapling device against one or more stapling locations.

### FIELD OF THE INVENTION

The present invention relates generally to an endovascular stapler delivery system employed in the treatment of vascular disease.

### BACKGROUND OF THE INVENTION

Grafting procedures have been used to treat aneurysms, such as aneurysms of the abdominal aorta and of the descending thoracic aorta. Aneurysms result from weak blood vessel walls that balloon due to aging and disease and pressure in the vessel. In addition, aneurysmal vessels have a potential to rupture, causing internal bleeding and potentially life threatening conditions. Grafts are used to isolate aneurysms or other blood vessel abnormalities from normal blood pressure, reducing pressure on the weakened vessel wall and reducing the chance of vessel rupture. A tubular endovascular graft is placed within the aneurysmal blood vessel to create a new flow path and an artificial flow conduit through the aneurysm, thereby reducing if not nearly eliminating the exertion of blood pressure on the aneurysm. The graft typically incorporates or is combined with one or more radially expandable stent(s) to be radially expanded in situ to anchor the tubular graft to the wall of the blood vessel at sites upstream and downstream of the aneurysm. Thus, endovascular grafts are typically held in place by mechanical engagement and friction provided by the radial force of the self-expanding or balloon expandable stents. However in some instances, the stent(s) support structure may fail to establish an acceptable long term fixation with the blood vessel wall. In such an event, the graft may undergo undesirable migration or slippage, or blood may leak into the aneurysmal sac, often referred to as an “endoleak”. To reduce the chance of migration, it may be desirable to fix a newly implanted graft using staples as the primary fixation method.

Endovascular staplers or stapling devices have shown effectiveness in preventing undesired graft migration. To deliver staples to secure the graft to the vessel wall, a stapling device is positioned within a luminal anatomical structure, e.g., a blood vessel or other anatomical conduit, for the purpose of attaching the endoluminal graft or other apparatus to the wall of the anatomical structure. A displacement or biasing member, such as a balloon structure, may be deployed to forcibly press the stapling device against a receiving area, i.e., the vessel wall, where a staple is to be fired to ensure that a fired staple will engage both the graft and the vessel wall. However, some known displacement members, such as balloons, may occlude blood flow during the procedure. In addition, in order for the displacement member to effectively press the stapling device against the receiving area where a staple is to be fired, it is required that the displacement member be properly aligned by the operator within the vessel, which can be a difficult endeavor. Thus, a need exists in the art for a guidance and alignment device that can deliver an endovascular stapler or stapling device to a treatment site such that the stapling device is properly aligned within a body vessel and positioned against a receiving area of a tubular prosthesis to be stapled.

### SUMMARY OF THE INVENTION

Embodiments described herein relate to an endovascular stapler or stapling device delivery apparatus for delivering a stapler or stapling device to a body vessel. The apparatus includes a continuous delivery rail slidably received within the lumen of a catheter (or removeable delivery sheath). The delivery rail is operable to guide the stapling device through the body lumen to a treatment site. The delivery rail includes an elongated first leg member extending in parallel with an elongated second leg member, and a self-expanding distal loop extending between distal ends of the first and second leg members. The distal loop has an expanded configuration that extends at an acute angle from the first and second leg members and includes a first contact portion along the distal loop that includes a first stapling location configured to abut the wall of the body vessel and a second contact portion along the distal loop that includes a second stapling location configured to abut the wall of the body vessel, wherein the first and second contact portions are longitudinally offset from each other. In an embodiment, the first and second contact portions are substantially diametrically opposed to each other about the distal loop such that each corresponds to an opposing side of the body vessel.

### BRIEF DESCRIPTION OF DRAWINGS

The foregoing and other features and advantages of embodiments hereof will be apparent from the following description as illustrated in the accompanying drawings. The accompanying drawings, which are incorporated herein and form a part of the specification, further serve to explain the principles of embodiments hereof and to enable a person skilled in the pertinent art to make and use embodiments of the invention. The drawings are not to scale.

FIG. 1 is a schematic side view of an endovascular stapler delivery system in accordance with an embodiment hereof.

FIG. 2 is a cross-sectional view along line 2-2 of FIG. 1.

FIG. 3 is a schematic side view of the delivery rail of FIG. 1 removed from the sheath catheter.

FIG. 4 is a schematic side view of a delivery rail according to another embodiment.

FIG. 5 is a schematic perspective view of the distal loop of FIG. 1.

FIG. 6 is an end view of the distal loop of FIG. 1 positioned within a body lumen along line 6-6 of FIG. 5.

FIGS. 7-9 diagrammatically illustrate the steps of a method of delivering a stapling device within a blood vessel.

FIG. 10 is a schematic side view of a distal portion of a delivery rail having a distal loop according to another embodiment.

### DETAILED DESCRIPTION

Specific embodiments hereof are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The following detailed description is merely exemplary in nature. Although the description of embodiments hereof are in the context of treatment of blood vessels such as the coronary, carotid and renal arteries, the invention may also be used in any other body passageways where it is deemed useful.

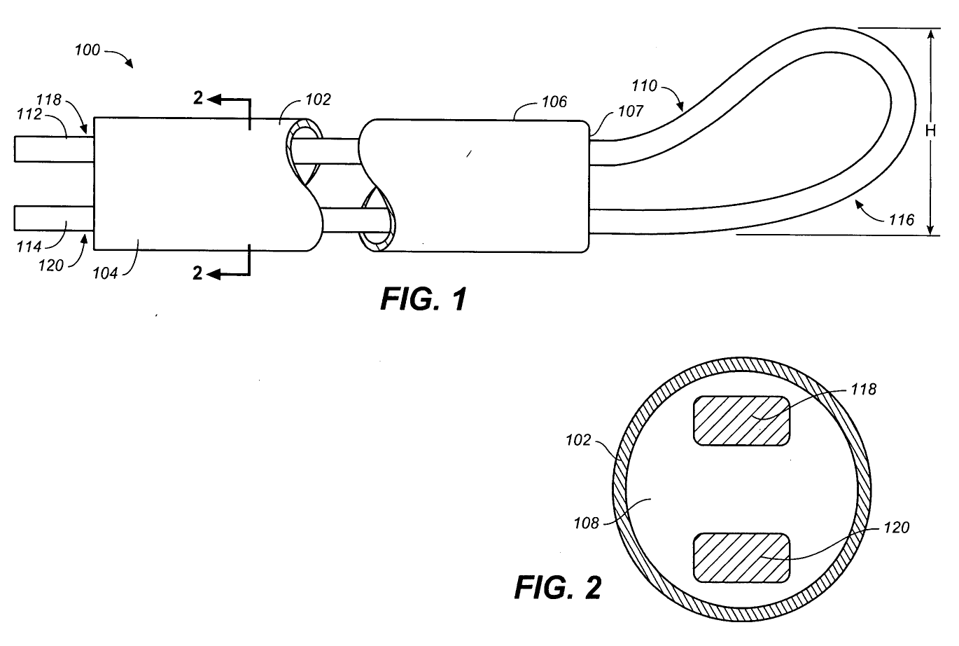

Embodiments described herein relate to a stapler or stapling device delivery system including a sheath or delivery catheter having a lumen extending therethrough and a continuous delivery rail slidably received within the lumen of the catheter. The delivery rail includes a first elongated leg, a second elongated leg, and a self-expanding distal loop, and is utilized as a track or guide to deliver a stapling device through the lumen of the catheter. In a deployed state, the distal loop abuts at least two opposing side walls of a body vessel. The stapling device may be guided around the distal loop such that the delivery rail allows for circumferential delivery of staples inside a body lumen. As the stapling device is delivered over the delivery rail, the distal loop pushes or forces the stapling device against one or more stapling locations, i.e., receiving areas where a staple is to be fired, to ensure that a fired staple will secure the graft to the vessel wall. Accordingly, the distal loop by design provides proper alignment of the stapling device within the vessel. In addition, during operation of the stapling device, the expanded distal loop may provide force to offset or counter firing of a staple to stabilize the stapling device and prevent or reduce movement during the firing of the staple. Further details and description of these embodiments are provided below with respect to FIGS. 1-10.

FIG. 1 is a schematic side view of an endovascular stapler or stapling device delivery system 100 in accordance with an embodiment hereof, with FIG. 2 showing a cross-sectional view of the system in FIG. 1 taken along line 2-2. System 100 includes a continuous delivery rail 110 slidably disposed within a lumen 108 of a sheath catheter 102. Sheath catheter 102 includes a proximal end 104 and a distal end 106, wherein distal end 106 includes an exit port 107. Lumen 108 of sheath catheter 102 is of a sufficient size to accommodate a stapling device. For example, a conventional stapling device typically has a profile or an outer diameter of approximately 4 mm-5 mm (12-15 French units) and thus the diameter of lumen 108 of sheath catheter 102 should be of a slightly larger size in order to ensure that a conventional stapling device can be advanced through sheath catheter 102. Another alternative would be that after the rail is delivered and in position, the sheath could be removed prior to advancement of stapler, so the radius of the sheath catheter needs to only be at least double the radius (+some margin) of the wire that makes up the rail. Lumen 108 may have any suitable cross-section, including a circular cross-section, as shown in FIG. 2, or an elliptical cross-section. It is desirable for lumen 108 to be as small as possible in order to minimize the outer diameter of sheath catheter 102, thus minimizing the crossing profile of stapler delivery system 100 such that it may fit within relatively small vessels.

Sheath or delivery catheter 102 may include an extruded shaft formed of any suitable flexible polymeric material. Non-exhaustive examples of material for the sheath catheter are polyethylene terephalate (PET), nylon, polyethylene, PEBAX, or combinations of any of these, either blended or co-extruded. Optionally, a portion of the sheath catheter may be formed as a composite having a reinforcement material incorporated within a polymeric body in order to enhance strength, flexibility, and/or toughness. Suitable reinforcement layers include braiding, wire mesh layers, embedded axial wires, embedded helical or circumferential wires, and the like. In an embodiment, the proximal portion of the sheath catheter may in some instances be formed from a reinforced polymeric tube, for example, as shown and described in U.S. Pat. No. 5,827,242 to Follmer et al. which is incorporated by reference herein in its entirety. The sheath catheter may have any suitable working length, for example, 55 cm-200 cm, in order to extend to a target location where a staple is to be fired.

Delivery rail 110 includes a first proximal end 112 and a second proximal end 114 that each extend proximally from proximal end 104 of sheath catheter 102 such that ends 112, 114 of delivery rail 110 extend out of the patient and may be manipulated by a clinician. Delivery rail 110 also includes a distal loop 116 that is positionable at the point of treatment. In FIG. 1, distal loop 116 of delivery rail 110 is shown protruding from exit port 107 of sheath catheter 102, but it should be understood that this is for illustrative purposes only and that during use while sheath catheter 102 is being advanced through the vasculature, delivery rail 110 would be maintained within lumen 108 of sheath catheter 102 until deployment. Delivery rail 110 is a continuous shaft or wire having a first elongated leg 118 extending from first proximal end 112 to distal loop 116, and a second elongated leg 120 extending from second proximal end 114 to distal loop 116, such that distal loop 116 extends between first leg 118 and second leg 120. Delivery rail 110 operates as a track for guiding a stapling device to one or more receiving areas of a vessel wall and/or graft where a staple is to be fired. Accordingly, an outer perimeter of delivery rail 110 is a relatively small dimension similar in one dimension to the outer diameter of a typical guidewire, which is about 0.382 mm (0.015 inch) to about 0.970 mm (0.038 inch). Cross sectional geometric asymmetry would make the a second cross sectional axis approximately 1.5 to 2 times larger.

In one embodiment, illustrated in FIG. 2, the cross-section of delivery rail 110 is rectangular, i.e., having a cross sectional length approximately 1.5 to 2 times the height dimension. As shown, the corners of square or rectangular delivery rail 110 may be slightly rounded for contacting the vessel wall in an atraumatic manner. The rectangular cross-section operates to position and hold the rotational orientation of the stapling device against one or more receiving areas of a vessel wall and/or graft where a staple is to be fired. More particularly, a side-firing stapling device may be advanced over delivery rail 110. Due to the corners of rectangular delivery rail 110, the stapling device is prevented from freely rotating around the perimeter of delivery rail 110. In another embodiment (not shown), delivery rail 110 may have alternative cross-sections known to those of ordinary skill in the art, including but not limited to triangular, circular, or elliptical.

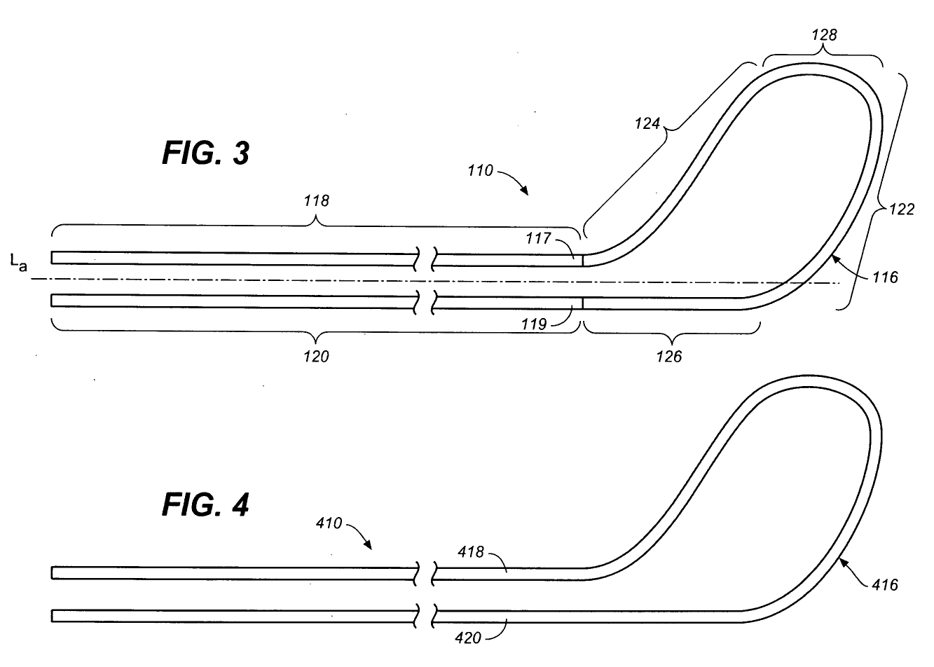

Delivery rail 110 is shown removed from sheath catheter 102 in FIG. 3. First and second elongated legs 118, 120 are generally straight segments that extend generally parallel to a longitudinal central axis Lₐ. Distal loop 116 is a curved segment, and is attached to the distal ends 117, 119 of first leg 118 and second leg 120 to form continuous delivery rail 110. Distal loop 116 may be attached to first and second legs 118, 120 in any suitable manner known in the art. For example, distal loop 116 may be attached via welding, such as by resistance welding, friction welding, laser welding or another form of welding such that no additional materials are used to connect distal loop 116 to first and second legs, 118,120. Alternatively, distal loop 116 and first and second legs 118, 120 can be connected by soldering, by the use of an adhesive, by the addition of a connecting element there between, or by another mechanical method.

Distal loop 116 is transformable between an unexpanded or delivery configuration (not shown) to an expanded or deployed configuration shown in FIG. 1. Distal loop 116 of delivery rail 110 is self-expanding meaning it has a mechanical memory to return to the expanded configuration. Mechanical memory may be imparted to distal loop 116 by thermal treatment to achieve a spring temper in stainless steel, for example, or to set a shape memory in a susceptible metal alloy, such as nitinol. In its delivery configuration, sheath catheter 102 surrounds and mechanically deforms distal loop 116 into a straightened configuration to minimize the delivery profile of system 100, which eases advancement of system 100 through the vasculature to the treatment site within a body vessel. When it is desired to deploy distal loop 116, sheath catheter 102 and delivery rail 110 may be moved relative to each other such that distal loop 116 is released from sheath catheter 102 and allowed to assume its expanded configuration. To cause the relative motion between sheath catheter 102 and delivery rail 110, delivery rail 110 may be distally advanced while sheath catheter 102 is held in place so that distal loop 116 is essentially pushed out of exit port 107, or sheath catheter 102 may be retracted in a proximal direction while delivery rail 110 is held in place so that distal loop 116 is essentially exposed, or a combination thereof. Once distal loop 116 exits sheath catheter 102, distal loop 116 elastically assumes its expanded configuration.

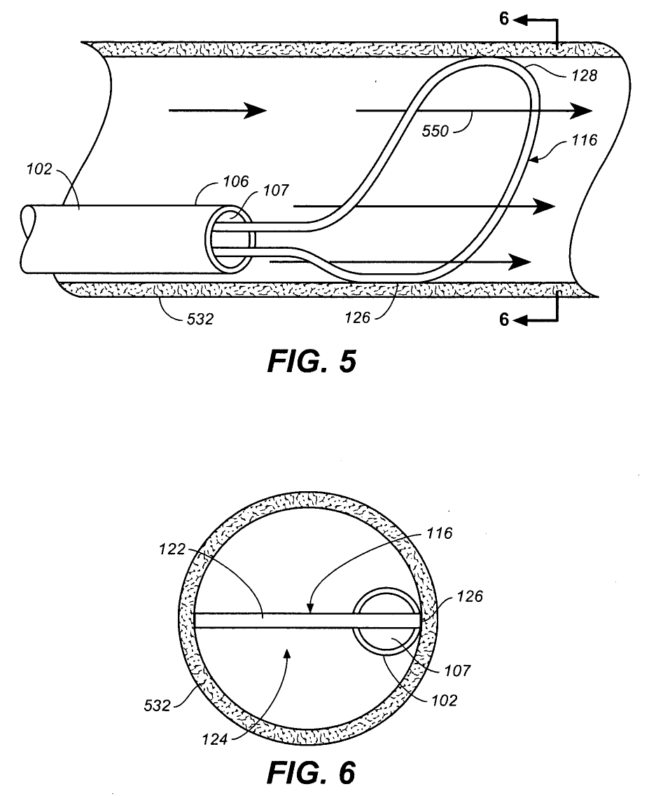

When expanded, a circumference of distal loop 116 of delivery rail 110 abuts and by design aligns with opposing side walls of a vessel. As may best be seen in the schematic perspective view of FIG. 5, expanded distal loop 116 assumes a skewed or angled oval-like shape when in place in situ within body vessel 532, wherein blood flow direction is represented by arrows 550 or a direction 180 degrees to the arrows 550. More particularly, at least a first contact portion 126 along distal loop 116 is configured to abut a vessel at a first stapling location and at least a second contact portion 128 along distal loop 116 is configured to abut the vessel at a second stapling location on the opposing or opposite side wall of the vessel. First and second contact portions 126, 128 of distal loop 116 each include a stapler location thereon that corresponds to the first or second stapling location, respectively. In the present embodiment, expanded distal loop 116 protrudes from sheath catheter 102 and first and second legs 118, 120 at an acute angle relative to longitudinal central axis Lₐ such that first portion 126 is longitudinally offset or staggered from second portion 128. Stated another way, first portion 126 is substantially located proximal of second portion 128. As will be explained in more detail herein, such an angled configuration of distal loop 116 provides particularly beneficial alignment and vessel wall contact in the area of the renal arteries, as when the renal arteries are offset from each other. A deployment height H shown in FIG. 1 of the expanded distal loop 116 must be sufficient to enable the expanded distal loop to abut opposing side walls of a vessel at points that are longitudinally offset from one and other as shown in FIG. 5. In an embodiment, deployment height H of the expanded distal loop 116 is of a slightly larger dimension than the diameter of the target vessel in order to ensure that the expanded distal loop abuts opposing side walls of a vessel. Accordingly, deployment height may depend on the diameter of the target vessel and thus may vary according to application.

When placed within a tubular anatomical conduit such as a blood vessel 532 shown in FIGS. 5 and 6, distal loop 116 is shown to provide a continuous track for guiding a stapling device along a circumference of the vessel. First and second portions 126,128 described above may be considered radially opposed and longitudinally offset portions of the continuous track that abut opposing or opposite sides of the vessel wall. In addition, distal loop 116 includes a first connector segment 122 that connects first portion 126 to second portion 128 and a second connector segment 124 that connects second portion 128 to a distal end of first leg 118, such that continuous distal loop 116 includes first and second portions 126, 128 and first and second connector segments 122, 124. In one embodiment, as illustrated the end view of FIG. 6, first and second connector segments 122, 124 abut opposite sides of vessel 532. First and second connector segments 122, 124 may make little to no contact with the vessel wall. Delivery rail 110 thus allows for circumferential delivery of staples inside a body lumen when the guiding rail distal loop 116 is re-set at a different rotational angle within the vessel and the stapling device is repeatedly guided to the stapling location.

In the embodiment of FIG. 3, first and second elongated legs 118, 120 may be formed from a material different than that of the self-expanding material of distal loop 116. For example, it may be desirable to form first and second legs 118, 120 from a material more rigid than the material used for distal loop 116. Suitable metallic materials for use in forming first and second legs 118, 120 include stainless steel, nickel-cobalt alloy such as MP35N, and cobalt-chromium. First and second legs 118, 120 may be solid core wires formed from a material such as stainless steel to impart improved pushability to delivery rail 110. In addition, core wires that form first and second legs 118, 120 may be ground down to have decreasing perimeters as each extends distally in order to provide a transition in stiffness along the length of delivery rail 110. The legs 118, 120 could be polymeric or metallic tubing. Well known polymers could be used, especially shape-memory polymers. Metallic tubing provides a geometrically non-symmetric cross-section that can be used to guide the stapler so that the staple is on the outside since the cross-sections of the stapler lumen and rail would fit like a lock & key.

In another embodiment shown in FIG. 4, delivery rail includes a distal loop 416 that is not a separate component from first and second legs 418, 420. In other words, delivery rail 410 is a unitary structure formed out of a single piece of material such that distal loop 416 is integrally formed with first and second legs 418, 420. In one embodiment, only the distal loop 416 is heat treated in order to impart a mechanical memory thereto such that distal loop 416 may achieve the expanded configuration.

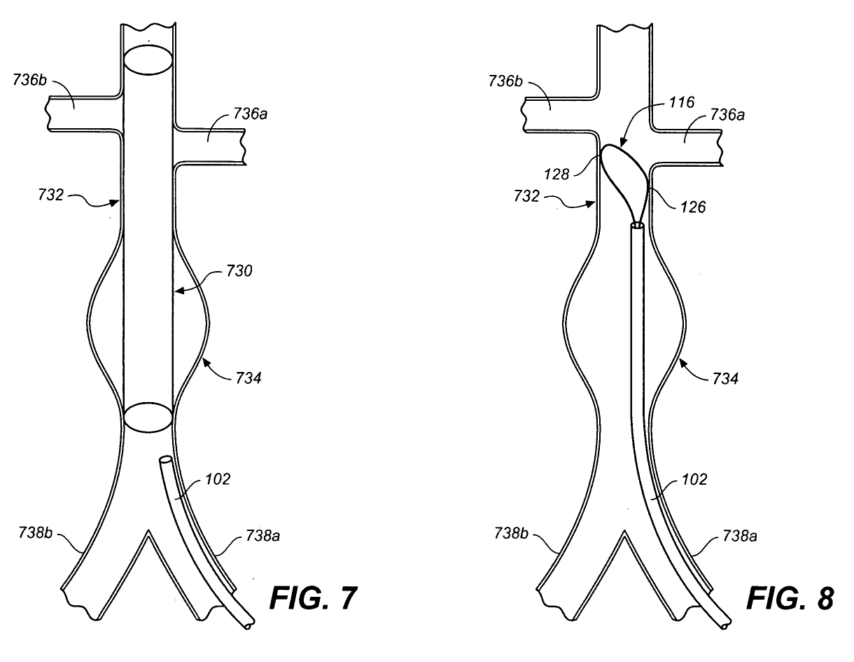

A method of delivering a stapling device within an aneurysm 734 according to an embodiment hereof is described with reference to FIGS. 7-9. The following method of delivering a stapling device is described to secure an endoluminal graft 730 within the abdominal aorta 732 in the location of renal arteries 736a, 736b, but it will be understood that the method may be utilized for delivering a stapling device to secure a graft or any other structure within other vasculature areas, including but not limited to the femoral artery, the thoracic branch arteries, and the renal arteries. The deployment height and shape of the expanded distal loop of the delivery rail may vary according to application. In addition, although methods of using specific embodiments are described herein for securing an endoluminal graft to a vessel wall, it will be apparent to those of ordinary skill in the art that such embodiments may also be utilized for securing extraluminal or transluminal grafts to a vessel wall.

FIG. 7 illustrates sheath catheter 102 being delivered proximal to the treatment site where graft 730 is implanted for treating abdominal aortic aneurysm 734. Access to the vasculature may be achieved through a branch of the femoral artery as shown, or alternatively, may be achieved through a carotid artery via an auxiliary artery. Methods and apparatus for delivering sheath catheter 102 intravascularly are generally known in the art and may be used to place and deliver sheath catheter 102 within the vasculature at the deployment site. In general, a guidewire (not shown) is introduced into the target vessel. Catheter 102 is then tracked over the guidewire such that the exit port 107 is adjacent to the implanted endoluminal graft 730. Once catheter 102 is in place as desired, the guidewire may be removed and delivery rail 110 may be slidably inserted through lumen 108 of catheter 102. Distal loop 116 of the delivery rail is held in an unexpanded configuration within catheter 102 as delivery rail 110 is tracked to the deployment site therethrough. Alternatively, delivery rail 110 may be pre-loaded within lumen 108 as sheath catheter 102 is tracked over the guidewire. Accordingly, lumen 108 of sheath catheter 102 may be sized to accommodate housing delivery rail 110 while being slidably tracked over the guidewire during delivery. In another embodiment, sheath catheter 102 may be constructed with a second guidewire lumen for housing the guidewire, as would be apparent to one of skill in the art, such that the delivery rail may be separately located within lumen 108 during delivery.

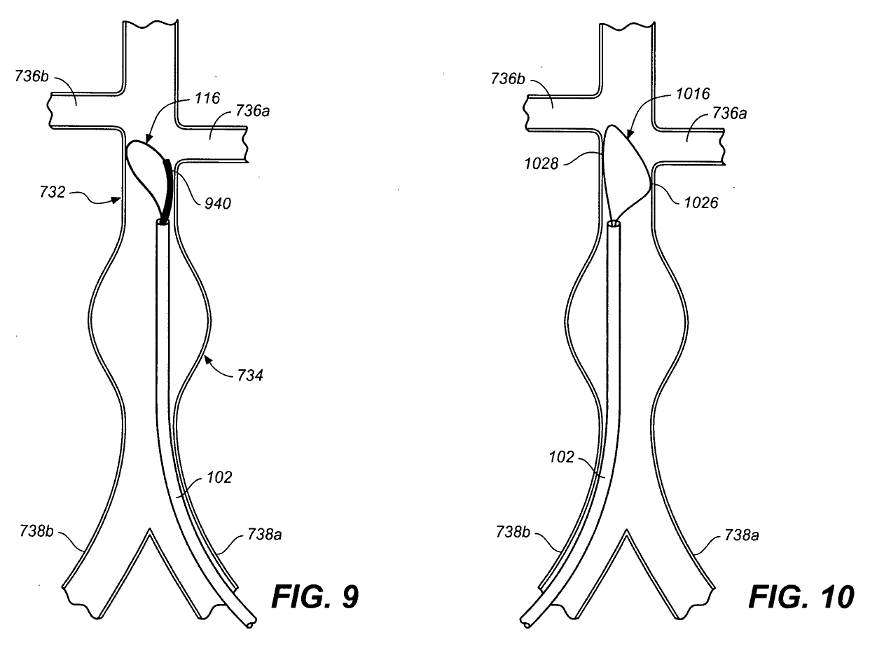

For sake of clarity, graft 730 has been removed in the schematic illustrations of FIGS. 8-9 such that it should be understood that delivery rail 110, and particular distal loop 116, are positioned within graft 730. When it is desired to deploy distal loop 116, catheter 102 and delivery rail 110 are moved relative to each other such that distal loop 116 is released from catheter 102 and allowed to assume its expanded configuration as described above. When expanded as shown in FIG. 8, distal loop 116 of delivery rail 110 is configured to abut and by design align with opposing side walls of aorta 732. More particularly, first portion 126 along distal loop 116 is configured to abut aorta 732 at a first location directly beneath the ostium of renal artery 736a and second portion 128 along distal loop 116 is configured to abut aorta 732 at a second location directly beneath the ostium of renal artery 736b on the opposing or opposite side wall of aorta 732. The angled configuration of distal loop 116, as described above, serves to align a stapling device such that staples may be fired at the first and second locations adjacent to the renal arteries and accommodates an anatomy wherein renal arteries 736a, 736b are offset from each other.

In FIGS. 7-9, renal artery 736b is shown at a slightly “higher” location along aorta 732 than opposing renal artery 736a. However, it is to be understood that individual anatomies differ and renal artery 736a may alternatively branch off at a slightly higher location along aorta 732 than opposing renal artery 736b. In either case, sheath catheter 102 is to be delivered through a branch 738a, 738b of the femoral artery corresponding to the “lower” renal artery. The angled configuration of distal loop 116, as described above, is configured to align a first proximal portion, such as first portion 126, along distal loop 116 directly beneath the “lower” renal artery and a second distal portion, such as second portion 128, along distal loop 116 directly beneath the “higher” renal artery on the opposing side wall of the aorta. Thus, in the embodiment of FIGS. 7-9, since renal artery 736a is the “lower” renal artery, sheath catheter 102 is shown as being delivered through femoral artery branch 738a.

Referring now to FIG. 9 with reference to the detail of delivery rail 110 in FIG. 3, a conventional stapling device 940 is inserted through sheath catheter 102 and tracked over second leg 120 of delivery rail 110 to first portion 126 along distal loop 116. Expanded distal loop 116 pushes or forces stapling device 940 against a first stapling location along aorta 732 directly beneath the ostium of renal artery 736a. Stapling device 940 is thus delivered over delivery rail 110 in an over-the-wire fashion. Since distal loop 116 of delivery rail 110 spans across and abuts opposing sides of aorta 732, distal loop 116 may aid in stabilizing the stapling device to prevent movement during the firing of a staple. Once a first staple is delivered, stapling device 940 may then be guided around distal loop 116 along first connector segment 122 to second portion 128. The stapling device is in position for the firing of a second or subsequent staple at a second stapling location directly beneath the ostium of renal artery 736b on the opposing or opposite side wall of aorta 732. It should be apparent from the description herein that additional staples may be delivered anywhere along first and second segments 122, 124, and/or multiple staples may be delivered at each stapling location. Once all the staples have been delivered and graft 730 is secured as desired, the stapling device 940 is retracted and removed from the patient. Distal loop 116 may then be retracted into sheath catheter 102 to collapse distal loop 116 into the delivery configuration, and the stapler delivery system removed from the patient. Although stapling device 940 is described as being tracked over second leg 120 of delivery rail 110 in FIG. 3, such that the stapling device initially reaches first portion 126 along distal loop 116, it should be understood that stapling device 940 may alternatively be tracked over first leg 118 in FIG. 3, such that the stapling device initially reaches second portion 128 along distal loop 116.

Embodiments described may be used with any conventional stapling device capable of being delivered in an over-the-wire fashion. Thus, it will be apparent to those of ordinary skill in the art that any features of the stapling device discussed herein are exemplary in nature. For example, the stapling device may be any stapling device known in the art, including but not limited to those shown or described in U.S. Patent Appl. Pub. No. 2004/0176786 assigned to Edrich Vascular, U.S. Patent Appl. Pub. No. 2007/0073389 assigned to Aptus Endosystems, Inc., and U.S. Patent Appl. Pub. No. 2007/0162053 assigned to Anson Medical.

The distal loop of the delivery rail may have alternative configurations from the angled oval-like loop described above. For example, in another embodiment shown in FIG. 10, expanded distal loop 1016 is shown protruding from sheath catheter 102 in a triangular shape such that a first portion 1026 along distal loop 1016 is longitudinally offset or located proximal to a second portion 1028 along distal loop 1016. The triangular shape of distal loop 1016 also provides particularly beneficial alignment in the area of renal arteries 736a, 736b when the renal arteries are offset from each other. However, unlike the angled oval-like embodiment, distal loop 1016 is to be delivered through a branch 738a, 738b of the femoral artery corresponding to the “higher” renal artery. The triangular configuration of distal loop 1016 as described above is configured to align second distal portion 1028 along distal loop 1016 directly beneath the “higher” renal artery and first proximal portion 1026 along the distal loop 1016 directly beneath the “lower” renal artery on the opposing side wall of the aorta. Thus, in the embodiment of FIG. 10, since renal artery 736b is the “higher” renal artery, sheath catheter 102 is shown as being delivered through femoral artery branch 738b.

While various embodiments have been described above, it should be understood that they have been presented by way of illustration and example only, and not limitation. It will be apparent to persons skilled in the relevant art that various changes in form and detail can be made therein without departing from the spirit and scope thereof. It will also be understood that each feature of each embodiment discussed herein, and of each reference cited herein, can be used in combination with the features of any other embodiment. All patents and publications discussed herein are incorporated by reference herein in their entirety.

What is claimed is:

1. An endovascular stapler delivery apparatus for delivering a stapling device to a body vessel, the apparatus comprising:
a continuous delivery rail for tracking the stapling device through the body vessel and aligning the stapling device at a stapling location, the delivery rail including
an elongated first leg member extending substantially parallel with a longitudinal axis of the stapler delivery apparatus,
an elongated second leg member extending substantially parallel with the first leg member, and
a self-expanding distal loop extending between a distal end of the first leg member and a distal end of the second leg member,
wherein the distal loop in an expanded configuration extends from the first and second leg members at an acute angle, such that a first contact portion along the distal loop defines a first stapler location and a second contact portion along the distal loop defines a second stapler location, wherein the first and second contact portions of the distal loop are longitudinally offset from each other.

2. The apparatus of claim 1, wherein the first and second contact portions are substantially diametrically opposed to each other about the distal loop and separated by a connecting segment.

3. The apparatus of claim 2, wherein the connecting segment includes at least an additional stapler location.

4. The apparatus of claim 1, wherein the self-expanding distal loop is a separate component attached at a first end to the distal end of the first leg member and at a second end to the distal end of the second leg member.

5. The apparatus of claim 1, wherein the first and second leg members of the delivery rail are formed from a first material and the distal loop is formed from a second material that is different from the first material.

6. The apparatus of claim 1, wherein the self-expanding distal loop and the first and second leg members of the delivery rail are integrally formed from a single material.

7. The apparatus of claim 1, wherein the first and second leg members each include a proximal end that proximally extends from the body vessel, such that the stapling device is trackable through the body vessel to the stapling location over either of the first and second leg members.

8. The apparatus of claim 1, further comprising:
a sheath catheter that surrounds and mechanically deforms the distal loop of the delivery rail into an unexpanded configuration for delivery to the stapling location within the body vessel.

9. The apparatus of claim 7, wherein relative sliding motion between the sheath catheter and the delivery rail releases the distal loop from the sheath catheter such that the distal loop self-expands into the expanded configuration.

10. The apparatus of claim 1, wherein the delivery rail has a rectangular cross-section.

11. A method of delivering a stapling device to a body vessel, the method comprising the steps:
tracking an endovascular stapler delivery apparatus to a target location within the body vessel, wherein the apparatus includes a continuous delivery rail having a first leg member extending in parallel with a second leg member, and having a self-expanding distal loop extending between the first leg and the second leg members;
expanding the distal loop to an expanded configuration, wherein the distal loop extends at an acute angle with respect to the first and second leg members such that at least a first contact portion along the distal loop abuts a wall of the body vessel at a first stapling location and a second contact portion along the distal loop abuts the wall of the body vessel at a second stapling location, the first and second stapling locations being on opposing sides of the vessel and longitudinally offset from each other;
tracking the stapling device over the first leg member of the delivery rail;
positioning the stapling device along the first contact portion of the distal loop at the first stapling location of the vessel; and
firing a staple from the stapling device into the wall of the vessel at the first stapling location.

12. The method of claim 11, wherein the staple secures a graft to the vessel.

13. The method of claim 11, further comprising:
tracking the stapling device around the distal loop to the second contact portion;
positioning the stapling device along the second contact portion at the second stapling location of the vessel; and
firing a staple from the stapling device into the wall of the vessel at the second stapling location.

14. The method of claim 11, wherein the first stapling location is along the abdominal aorta directly below a first renal artery and the second stapling location is along the abdominal aorta directly below a second renal artery.

15. The method of claim 11, further comprising:
providing a sheath catheter to surround and mechanically deform the distal loop of the delivery rail into an unexpanded configuration during delivery of the stapler delivery apparatus to the target location within the body vessel.

16. The method of claim 15, wherein the step of expanding the distal loop includes relative sliding motion between the sheath catheter and the delivery rail to allow the distal loop to assume the expanded configuration.

17. The method of claim 11, wherein the first and second contact portions of the distal loop are separated by a connector segment that abuts at least one additional stapling location.

18. The method of claim 17, further comprising:
tracking the stapling device along the connector segment of the distal loop;
positioning the stapling device along the connector segment at the additional stapling location; and
firing a staple from the stapling device into the wall of the vessel at the additional stapling location.

19. The method of claim 11, wherein the delivery rail has a rectangular cross-section.
